(19) United States
(12) Patent Application Publication
(10) Pub. No.: US 2017/0266156 A1
(43) Pub. Date: Sep. 21, 2017

(54) METHOD FOR PREVENTING CANCER BY USING PHTHALIDES

(71) Applicant: National Sun Yat-sen University, Kaohsiung (TW)

(72) Inventors: Chia-Chen Wang, Kaohsiung (TW); Wei-Ren Chen, Kaohsiung (TW)

(73) Assignee: National Sun Yat-sen University, Kaohsiung (TW)

(21) Appl. No.: 15/071,190

(22) Filed: Mar. 15, 2016

(51) Int. Cl. A61K 31/365; A61K 31/683

(52) U.S. Cl. CPC A61K 31/365; A61K 31/683

(57) ABSTRACT

The present invention relates to a method for preventing cancer by using a phthalide compound, wherein the phthalide compound has an effect of increasing the oxygen release efficiency of hemoglobin (Hb) of a subject to increase the oxygenation level of organs and tissue cells, thereby preventing the cellular oxygenation level against falling below the critical cellular oxygen requirements under which the normal cells can turn cancerous. Although cancer may be caused by a variety of reasons, including congenital inheritance, external environment, air pollution or poor living and dietary habits, there is only one primary and common reason in causing cancers, the excessively low cellular oxygenation level. When the oxygenation level of any cell falls below 60% of its physiological oxygen requirements, the normal cell may turn cancerous. The present invention is to use the phthalide compound to substitute for or cooperate with 2,3-BPG of a subject to lower the oxygen affinity of hemoglobin (Hb) and to facilitate the release of oxygen from hemoglobin to tissue cells, as a result, the cellular oxygenation level is maintained always higher than the critical threshold for the normal cells to turn cancerous, and by doing so to prevent the normal cells from turning cancerous and prevent the developments of cancers.

### FIELD OF THE INVENTION

The present invention is in the medical field, relating to a method for preventing cancers by using a phthalide compound.

### BACKGROUND OF THE INVENTION

Cancer may be caused by a variety of reasons, including congenital inheritance, external environments, air pollution or even poor living or dietary habits. Though these carcinogenic factors may seem irrelevant from each other, however they intrinsically share a common reason in being carcinogenic, i.e., the excessively low oxygenation level of tissue cells.

Otto Warburg, the laureate Nobel Prize in Physiology or Medicine in 1931, pointed out as early as in the 1930s that when the oxygenation level of any normal cell falls below 60% of its oxygen requirements, it turns cancerous. As shown in FIG. 9, a tumor-bearing mouse has significantly lower PO₂ in the tumor region than the same region of a healthy mouse, and the PO₂ in the non-tumor region of the tumor-bearing mouse is significantly lower than the same region of a healthy mouse.

Even though cancer has long been regarded as a congenital hereditary disease, however, more and more scientific evidence shows that cancer is in fact a metabolic disease. A variety of environmental factors, including outdoor air pollution and suspended particulate matters therein, and processed food are all confirmed to be carcinogens in recent years. Outdoor air pollution and aerosol particles were officially declared as carcinogens by the World Health Organization (WHO) in 2013, among which the PM2.5 aerosol particles, defined as the particulate matters having an aerodynamic diameter less than 2.5 micrometers are the most hazardous to health. In addition, in 2015, WHO officially declared processed meat as a carcinogen, which may easily cause colorectal cancer, pancreatic cancer and prostate cancer, etc. These carcinogens are often rich in highly oxidizing chemical species, for example reactive oxygen species (ROS) and reactive nitrogen species (RNS), which may interact with hemoglobin (Hb) to cause oxidative damages to hemoglobin (Hb) and thus modify the structure of hemoglobin (Hb), thereby decreasing the oxygen transport efficiency and resulting in an reduced oxygenation level of tissue cells. Once the oxygenation level of tissue cells becomes lower than 60% of its normal oxygen requirements, malignant lesions may occur and turn into cancerous cells.

Therefore, when the cellular oxygenation level is maintained always above 60% of its normal oxygen requirements, it is possible to prevent the normal cells against transforming into the cancer cells.

Hemoglobin (Hb), the oxygen-carrying protein in erythrocytes transports oxygen from respiratory organs such as respiratory tracts and lungs and releases oxygen to organs and peripheral tissues of a human body such that the organs and the peripheral tissues can be supplied with sufficient oxygen in order to maintain normal physiological functions.

Hemoglobin of human adults is a tetramer α₂β₂ consisting of four subunits, α₁, α₂, β₁ and β_{2,} wherein each subunit relies on intermolecular interactions such as intra-subunit hydrogen bonds to sustain its secondary and tertiary structures. Additionally, the inter-subunit hydrogen bonds formed among the aforementioned four subunits allow the quaternary structure of hemoglobin to be formed.

Hemoglobin can reside in two different quaternary structures, including the relaxed form (R form) having high oxygen affinity and the tense form (T form) having low oxygen affinity. When hemoglobin is travelled to lungs through the blood circulation, hemoglobin becomes bound with oxygen and resides in the R quaternary configuration of high oxygen affinity. The oxygenated hemoglobin is then transported to organs and peripheral tissues through blood circulation and releases oxygen to organs and peripheral tissues and transforms into the T quaternary configuration of low oxygen affinity. The allostery of hemoglobin is also influenced by several allosteric factors, such as the pH value, the carbon dioxide concentration and the 2,3-BPG concentration in erythrocytes.

2,3-bisphosphorglycerate (2,3-BPG or 2,3-diphosphoglycerate, 2,3-DPG, hereinafter “2,3-BPG”) is the endogenous allosteric effector of hemoglobin and the most important chemical species in an erythrocyte of a human body besides the oxygen-carrying entity, hemoglobin. 2,3-BPG delicately regulates the configuration of hemoglobin by interacting with the β₁ and β₂ subunits of hemoglobin to stabilize hemoglobin in the low oxygen affinity T form to reduce the oxygen affinity of hemoglobin, thereby facilitating hemoglobin to effectively release oxygen to body organs and tissue cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the synergistic effect of phthalide compounds and 2,3-BPG; A: Z-ligustilide; B: senkyunolide I.

FIG. 2 shows the oxygen equilibrium curves for hemoglobin (Hb) treated with various 2,3-BPG concentrations (0.2), showing that when the concentration of 2,3-BPG is higher, the oxygen equilibrium curve shifts more toward the right and the P₅₀ value is higher. The curves from the left to the right respectively represent: pure hemoglobin (Pure Hb) as the control group, 2,3-BPG, 1.2 mM 2,3-BPG, 4.0 mM 2,3-BPG, 8.0 mM 2,3-BPG and 12.0 mM 2, 3-BPG.

FIG. 3 shows the oxygen equilibrium curves of hemoglobin (Hb) treated with various 2,3-BPG concentrations (0.2-12 mM), illustrating how the oxygen saturation fraction of hemoglobin is altered at various oxygen partial pressures corresponding to human brain tissues, normal tissues and alveoli when hemoglobin (Hb) is modulated by 2,3-BPG. The curves from the left to the right respectively represent: pure hemoglobin (Pure Hb) as the control group, 0.6 mM 2,3-BPG, 1.2 mM 2,3-BPG, 4.0 mM 2,3-BPG, 8.0 mM 2,3-BPG and 12.0 mM 2, 3-BPG.

FIG. 4 shows that the P₅₀ value of hemoglobin (Hb) increases when concentration of the phthalide compounds increases, indicating that the oxygen affinity of hemoglobin (Hb) decreases and the oxygen release rate of hemoglobin (Hb) increases.

FIG. 5 shows that even at a low level of 2,3-BPG, various phthalide compounds can help to modulate hemoglobin (Hb) such that Hb can reach a comparable P₅₀ value as that of hemoglobin under a normal level of 2,3-BPG.

FIG. 6 shows that the oxygen equilibrium curves are modulated adjunctly by 2,3-BPG along with the phthalide compound, illustrating that the phthalide compound can cooperate with 2,3-BPG to decrease the blood oxygen saturation fraction and to increase the oxygen release efficiency when the oxygen partial pressure remains unchanged. The curves from the left to the right respectively represent: pure hemoglobin (Pure Hb) as the control group, 1.2 mM 2,3-BPG, 1.2 mM 2,3-BPG and 1.2 mM phthalide compound, and 1.2 mM 2,3-BPG and 4.0 mM phthalide compound.

FIGS. 7A-7L show the chemical structures of twelve phthalide compounds; 7A: Z-butylidenephthalide; 7B: Z-ligustilide; 7C: senkyunolide A; 7D: senkyunolide H; 7E: senkyunolide I; 7F: senkyunolide F; 7G: E-butylidenephthalide; 7H: E-ligustilide; 7I: 3-butylphthalide; 7J: 3-butylidene-4-hydrophthalide; 7K: 6,7-dihydroxyligustilide; 7L: 6,7-epoxyligustilide.

FIG. 8 is a diagram, showing the molecular structure of the functional groups of the phthalide compound.

FIG. 9 shows anatomical diagrams of a healthy mouse and the tumor region of a tumor-bearing mouse and diagrams of the PO₂ distribution for comparison; A: healthy mouse anatomical diagram; B: PO₂ of healthy mouse; C: tumor-bearing mouse anatomical diagram; D: PO₂ of tumor-bearing mouse; E: comparison of PO₂ for the healthy mouse, non-tumor-bearing region of the tumor-bearing mouse and the tumor-bearing region of the tumor-bearing mouse.

### SUMMARY OF THE INVENTION

The present invention relates to a method for preventing cancers by using a phthalide compound, wherein the phthalide compound can increase the oxygen-release efficiency of hemoglobin (Hb) of a subject, which further increases the oxygenation level of organs and tissue cells, thereby preventing the cellular oxygenation level from falling below the critical threshold cellular oxygen requirements, under which the normal cells may turn cancerous. Although cancers may be caused by a variety of reasons, including congenital inheritance, external environments, air pollution, or even poor living and dietary habits, there is only one primary and common reason, i.e., the excessively low cellular oxygenation level. When the oxygenation level of any cell falls below 60% of its physiological oxygen requirements, the normal cell may turn cancerous. The present invention uses the phthalide compound to substitute for or cooperate with 2,3-BPG of a subject to lower the oxygen affinity of hemoglobin (Hb) and thereby facilitating the oxygen release from hemoglobin to tissue cells, such that the cellular oxygenation level can be maintained constantly at a level always higher than the critical threshold under which the normal cells can turn cancerous, thereby preventing the tissue cells from turning cancerous.

### DETAILED DESCRIPTION OF THE INVENTION

The present invention uses a phthalide compound as a substitute of 2,3-BPG to facilitate the oxygen release efficiency of hemoglobin (Hb) to increase the oxygenation level of tissue cells, whereby the oxygenation level of various organs and peripheral tissue cells is controlled within a safe range to prevent them from transforming into cancerous cells. The present invention is to use a phthalide compound as a cancer prevention medicine, which can be applied to individuals who are evaluated as of high-risk in developing cancers.

The high-risk group whose members are prone to getting cancer comprises people who have a family history of cancer, who are constantly exposed to carcinogenic environments, and who frequently consume processed foods. Carcinogenic environments are environments containing many carcinogens, for example PM2.5 aerosol particles and air pollution factors.

The present invention closely and periodically monitors the oxygenation level of organs and tissue cells of those who are members of high-risk group for cancer, and provides preventive medication according to the oxygenation level needed to be increased in order to prevent the oxygenation level of organs and tissues cells from decreasing to a level lower than 60% of its physiological oxygen requirements, thereby inhibiting the transformation of normal cells into cancerous cells.

The primary purpose of the present invention is to provide a method for preparing a cancer prevention drug by using a phthalide compound, wherein the phthalide compound has the effect of increasing the oxygen release efficiency of hemoglobin (Hb) of a treated subject. The method of the present invention is to increase the oxygenation level of organs and tissue cells by increasing the oxygen release efficiency of hemoglobin (Hb), as a result, the oxygenation level of organs and tissues cells is prevented from decreasing to a level less than 60% of the physiological oxygen requirements, thereby inhibiting the transformation of normal cells into cancerous cells.

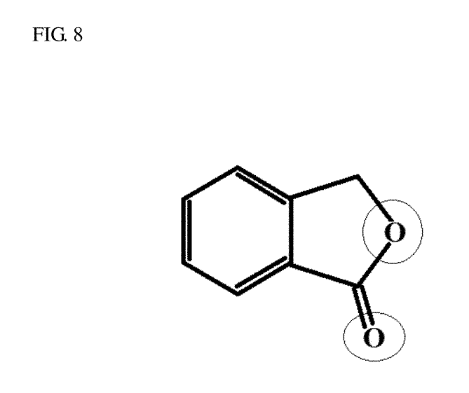

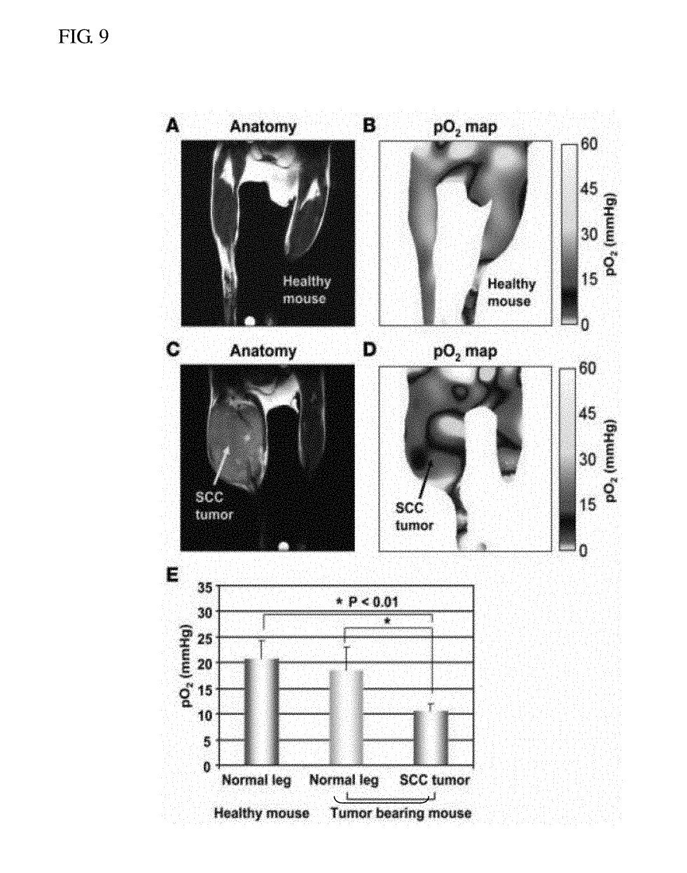

The phthalide compound of the present invention is any compound which has the structural characteristics of the functional groups of phthalide compounds as shown in FIG. 8, wherein the circled areas are the molecular structure of the functional groups of the phthalide compounds characterized by an endocyclic oxygen and an adjacent ketone.

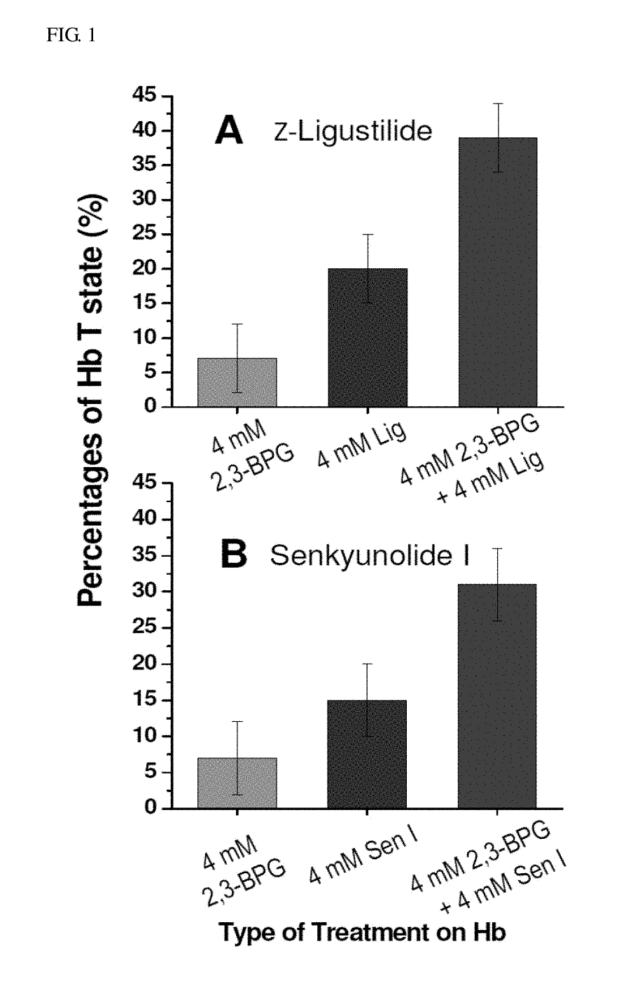

The phthalide compound of the present invention is not only used to substitute for or complement 2,3-BPG of a treated subject, but also to cooperate adjunctly with 2,3-BPG to have a synergistic effect in lowering the oxygen affinity of hemoglobin, thereby increasing the oxygen release efficiency of hemoglobin (Hb) (as shown in FIG. 1).

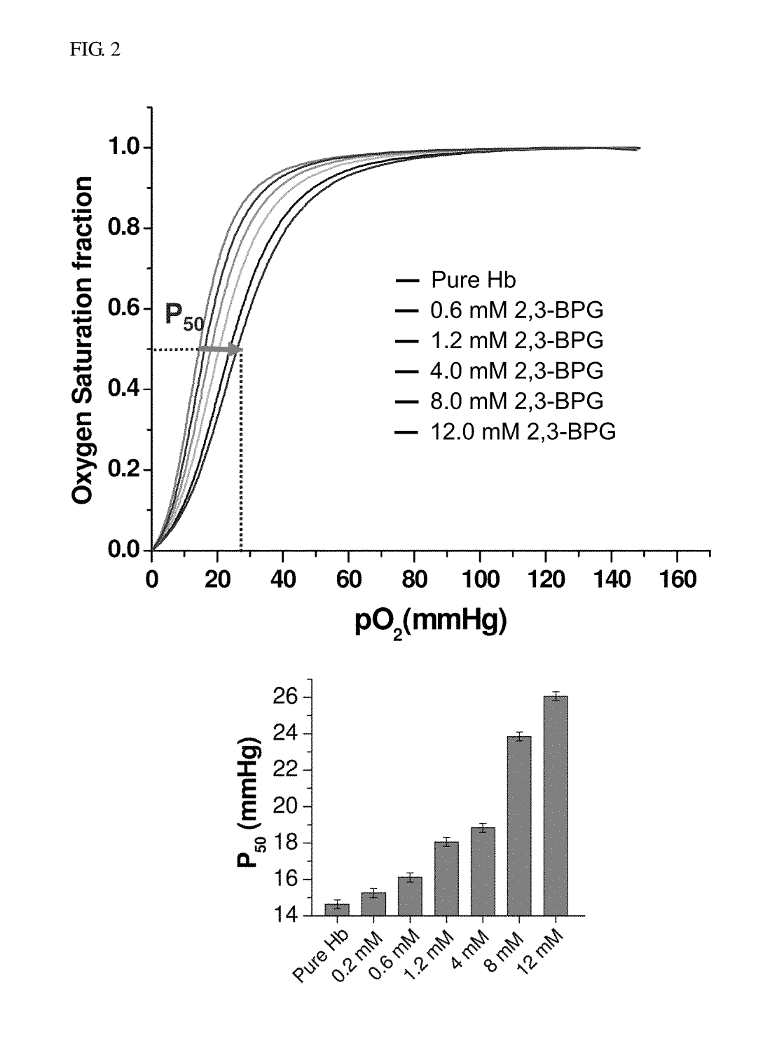

The oxygen affinity of hemoglobin (Hb) is commonly characterized by P₅₀ value. The P₅₀ value is the required oxygen partial pressure to achieve 50% oxygen saturation. The P₅₀ value of a normal adult is approximately 3.59 kPa (27 mmHg). An increased blood PCO₂, a decreased pH or an increased 2,3-BPG level in erythrocytes can all decrease the oxygen affinity of hemoglobin (Hb), so that the oxygen equilibrium curve shifts to the right and the P₅₀ value increases (as shown in FIG. 2); contrarily, when the oxygen affinity of hemoglobin (Hb) increases, the oxygen equilibrium curve shifts to the left and the P₅₀ value decreases.

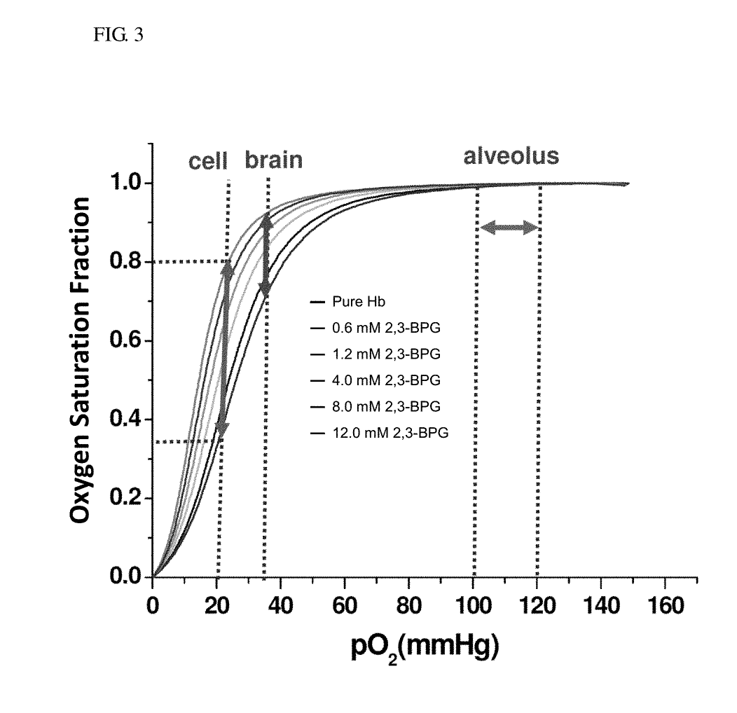

Under the normal physiological conditions, the PO₂ (oxygen partial pressure) of human cells is approximately 9.9-19 mmHg (J. Cell. Mol. Med., 15, 1239-1253 (2011)). By observing from the oxygen equilibrium curves of hemoglobin the effect of varying concentrations of 2,3-BPG on the oxygen saturation fraction of hemoglobin at a fixed oxygen partial pressure (as shown in FIG. 3), the effect of 2,3-BPG on increasing the oxygen release efficiency of hemoglobin (Hb) is explicitly revealed. For example, when hemoglobin is treated with 12 mM of 2,3-BPG (the purple curve shown in FIG. 3), at a fixed oxygen partial pressure of 20 mmHg, the oxygen saturation fraction of hemoglobin (Hb) decreases from 80% where no 2,3-BPG is present in hemoglobin (Hb) (the gray curve shown in FIGS. 3) to 35%, indicating that the oxygen release efficiency increases from 20% to 65%.

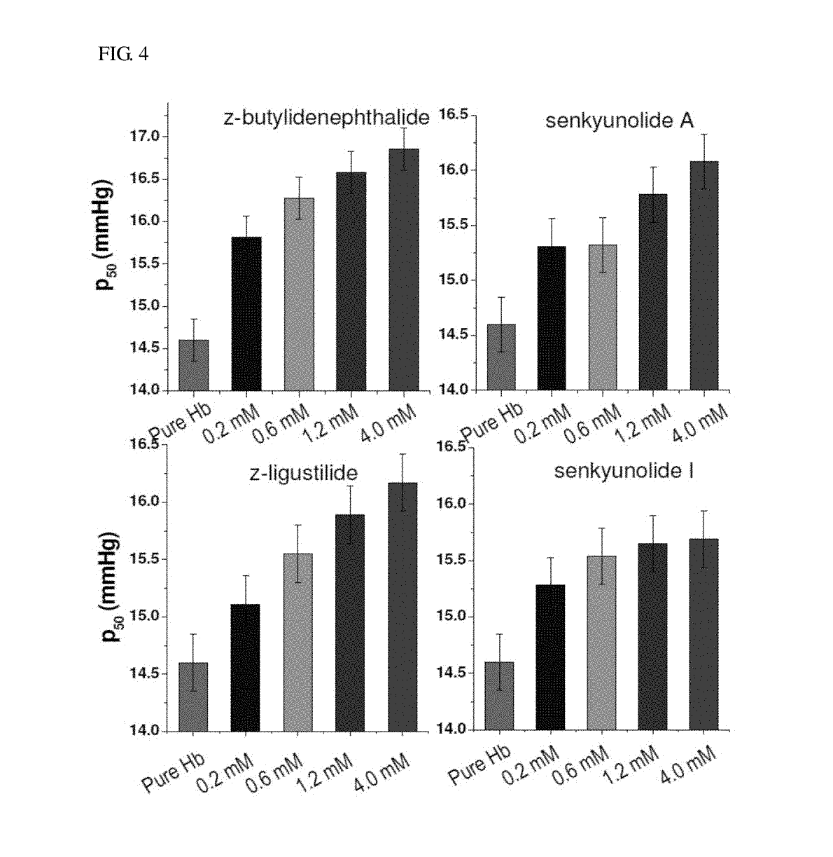

In one preferred embodiment, the phthalide compound can function as 2,3-BPG to effectively increase the P₅₀ value of hemoglobin (Hb), that is, to decrease the oxygen affinity of hemoglobin (Hb), and the higher the concentration of the phthalide compound, the higher the P₅₀ value and the lower the oxygen affinity are (as shown in FIG. 4).

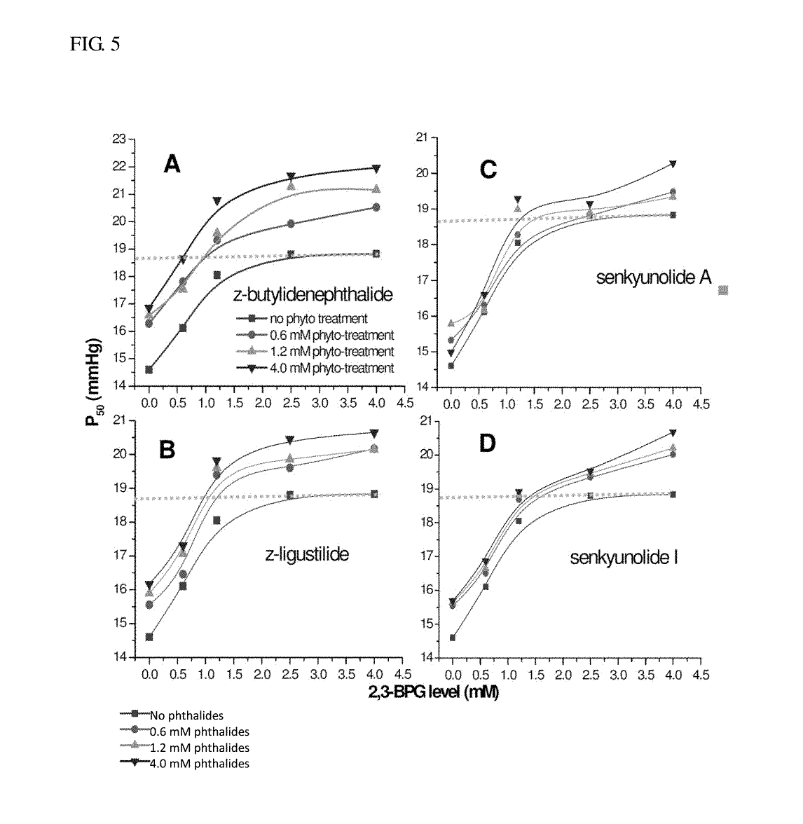

In another embodiment, when no phthalide compound is treated to hemoglobin, approximately 4 mM of 2,3-BPG is required for hemoglobin to achieve a P₅₀ value of 18.8 mmHg; but after hemoglobin is treated with a phthalide compound, only approximately 0.6-1.2 mM of 2,3-BPG is required to achieve a similar or higher P₅₀ value (as shown in FIG. 5).

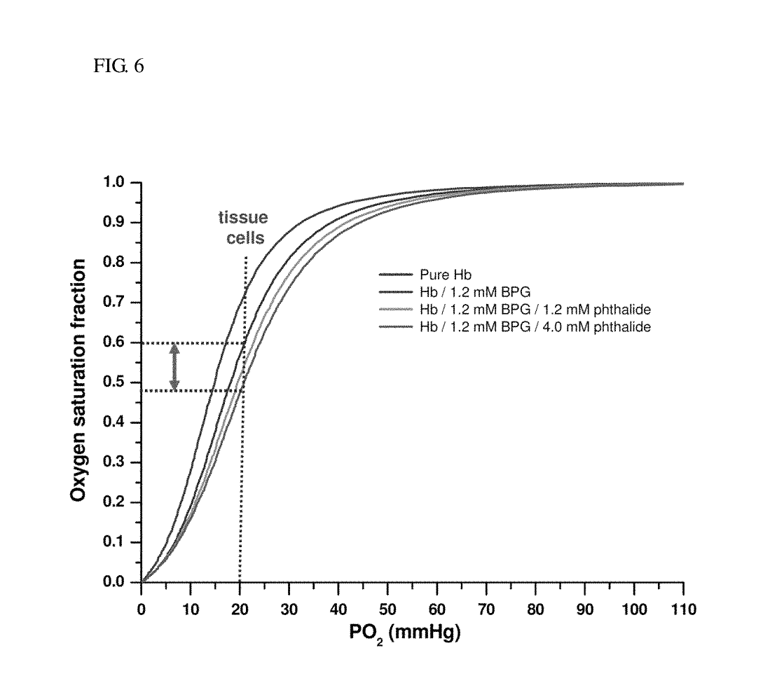

In another embodiment, as shown in FIG. 6, under 1.2 mM 2,3-BPG, the oxygen saturation fraction of hemoglobin at the oxygen partial pressure PO₂ of 20 mmHg is approximately 60%, but after an additional phthalide compound is administered, the oxygen saturation fraction of hemoglobin decreases from 60% to approximately 47%, indicating that the oxygen release efficiency of hemoglobin increases from 40% to 53%. Therefore, it confirms that the phthalide compound is able to act together with 2,3-BPG in the treated subject to facilitate hemoglobin (Hb) to release more oxygen at the same oxygen partial pressure.

The present invention provides a method for preparing cancer prevention drugs, wherein the phthalide compound has the effect on increasing the oxygen release efficiency of hemoglobin (Hb) in a treated subject. The present invention is to increase the oxygenation level of organs and tissue cells by increasing the oxygen release efficiency of hemoglobin (Hb), as a result the oxygenation level of organs and tissues cells is prevented from dropping to a level less than 60% of the physiological oxygen requirements, thereby inhibiting the transformation of normal cells into cancerous cells. The phthalide compound and 2,3-BPG have a synergistic effect in lowering the oxygen affinity of hemoglobin.

### EXAMPLES

The examples and figures mentioned in the following text are used to illustrate the technical content, characteristics and advantages of the present invention and are not used to limit the present invention.

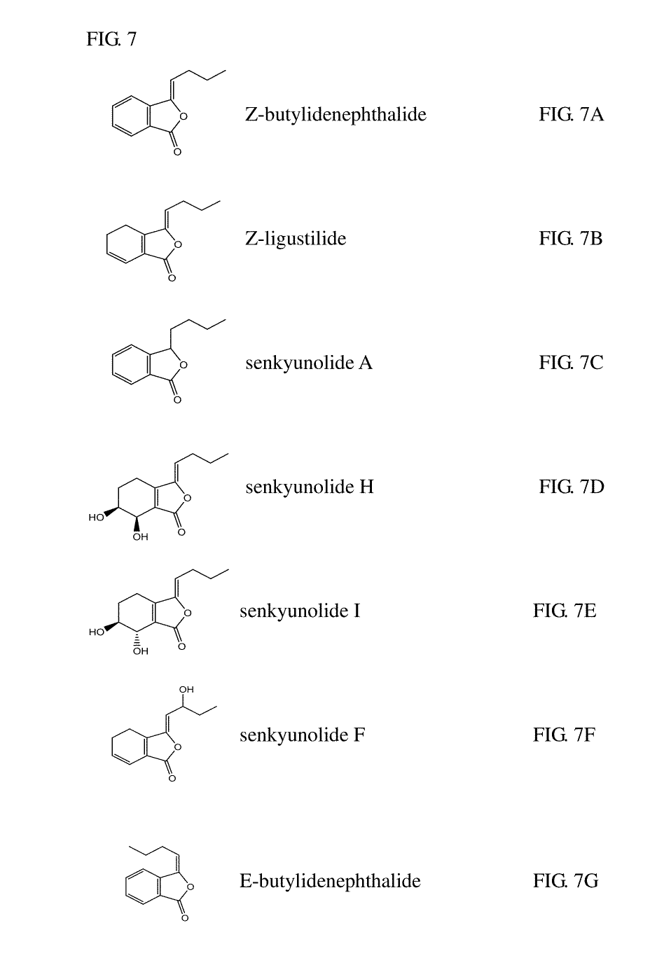

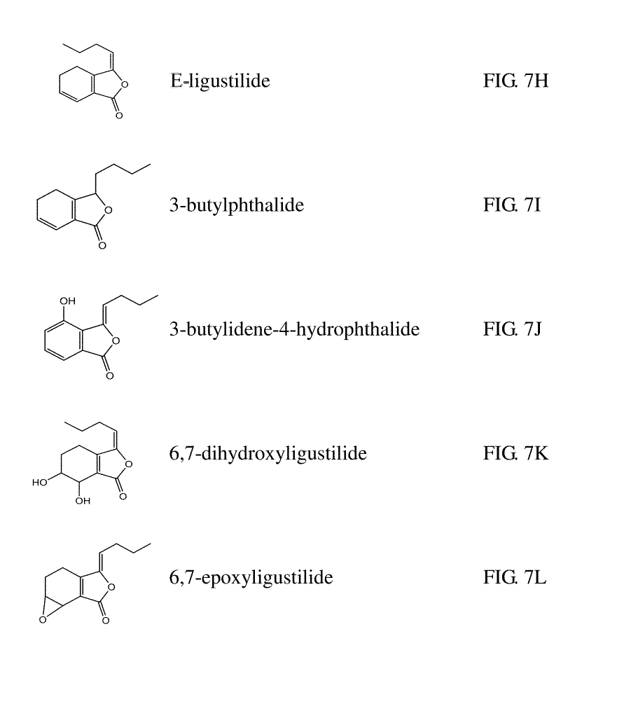

The phthalide compound provided by the present invention could be any compound comprising the structural characteristics of the phthalide compounds, such as Z-butylidenephthalide (as shown in FIG. 7A), Z-ligustilide (as shown in FIG. 7B), senkyunolide A (as shown in FIG. 7C), senkyunolide H (as shown in FIG. 7D), senkyunolide I (as shown in FIG. 7E), senkyunolide F (as shown in FIG. 7F), E-butylidenephthalide (as shown in FIG. 7G), E-ligustilide (as shown in FIG. 7H), 3-butylphthalide (as shown in FIG. 7I), 3-butylidene-4-hydrophthalide (as shown in FIG. 7J), 6,7-dihydroxyligustilide (as shown in FIG. 7K) and 6,7-epoxyligustilide (as shown in FIG. 7L).

The oxygen affinity of hemoglobin is commonly characterized by P₅₀ value. The P₅₀ value is the required oxygen partial pressure for hemoglobin to achieve 50% oxygen saturation. The P₅₀ value of a normal adult is approximately 3.59 kPa (27 mmHg). An increased blood PCO₂, a decreased pH or an increased concentration of 2,3-BPG in erythrocytes could all decrease the oxygen affinity of hemoglobin, as a result, the oxygen equilibrium curve shifted to the right and the P₅₀ value increased (as shown in FIG. 2); contrarily, when the oxygen affinity of hemoglobin increased, the oxygen equilibrium curve shifted to the left and the P₅₀ value decreased.

Under the normal physiological conditions, the PO₂ (oxygen partial pressure) of human cells is approximately 9.9-19 mmHg (J. Cell. Mol. Med., 15, 1239-1253 (2011)). By observing from the oxygen equilibrium curves of hemoglobin the effect of varying concentrations of 2,3-BPG on the oxygen saturation fraction of hemoglobin at a fixed oxygen partial pressure (as shown in FIG. 3), the effect of 2,3-BPG on increasing the oxygen release efficiency of hemoglobin (Hb) is explicitly revealed. For example, when 12 mM of 2,3-BPG is administrated to hemoglobin (the 6^{th} curve from the left, shown in FIG. 3), at a fixed oxygen partial pressure of 20 mmHg, the oxygen saturation fraction of hemoglobin (Hb) decreases from 80% where no 2,3-BPG is present in hemoglobin (Hb) (the first curve from the left, shown in FIGS. 3) to 35%, indicating that the oxygen release efficiency increases from 20% to 65%.

In one preferred example, the phthalide compound could function as 2,3-BPG to effectively increase the P₅₀ value of hemoglobin (Hb), that was, to decrease the oxygen affinity of hemoglobin (Hb), and the higher the concentration of the phthalide compound, the higher the P₅₀ value and the lower the oxygen affinity were (as shown in FIG. 4).

In another example, when no phthalide compound was treated to hemoglobin, approximately 4 mM of 2,3-BPG was required for hemoglobin to achieve a P₅₀ value of 18.8 mmHg; but after hemoglobin was treated with a phthalide compound, only approximately 0.6-1.2 mM of 2,3-BPG was required to achieve a similar or higher P₅₀ value (as shown in FIG. 5).

In another example, as shown in FIG. 6, under 1.2 mM 2,3-BPG, the oxygen saturation fraction of hemoglobin at the oxygen partial pressure PO₂ of 20 mmHg was approximately 60%, but after an additional phthalide compound was administered, the oxygen saturation fraction of hemoglobin decreased from 60% to approximately 47%, indicating that the oxygen release efficiency of hemoglobin increased from 40% to 53%. Therefore, it confirmed that the phthalide compound was able to act together with 2,3-BPG in the treated subject to allow hemoglobin (Hb) to release more oxygen when the oxygen partial pressure remained unchanged, as a result, the oxygenation level of tissue cells is increased, thereby preventing the cellular oxygenation level from dropping to a level lower than the critical threshold, i.e., 60% of the normal oxygen requirements below which the normal cells may turn cancerous.

In one example, the phthalide compound could be used together with other compounds which were capable of stabilizing the oxygen-carrying hemoglobin (Hb) in the T form and effectively decreasing the oxygen affinity of hemoglobin (Hb) to increase the oxygen release efficiency of the hemoglobin (Hb) of a treated subject.

In another example, the method could co-administer 2,3-BPG and a drug prepared by using the phthalide compound to a subject in need thereof, wherein the methods for administering the drug comprised injection.

In another example, besides the phthalide compound, 2,3-BPG was also administered to a subject in need thereof.

In another example, the method could co-administer 2,3-BPG and a drug prepared by using the phthalide compound to a subject in need thereof, wherein the methods for administering the drug comprised injection.

In another example, the drug further comprised 2,3-BPG.

In one example, the methods for administering the drug prepared by using the phthalide compound comprised: oral administration, injection and inhalation as aerosolized medication to increase the oxygen release efficiency of hemoglobin (Hb) of the treated subject. In another example, the drug could be administered in combination with other cancer treatments to a subject as a method of auxiliary treatment, wherein the method for administering the drug comprised oral administration, injection and inhalation as aerosolized medication.

In summary, the method of the present invention is to provide a method for preventing cancer by using a phthalide compound, wherein the phthalide compound has the effect of increasing the oxygen release efficiency of the hemoglobin (Hb) of a treated subject. The present invention is to increase the oxygenation level of organs and tissue cells by changing the oxygen release efficiency of hemoglobin (Hb), as a result, the oxygenation level of organs and tissues cells is prevented from dropping to a level less than 60% of the oxygen requirements such that the transformation of normal cells into cancerous cells can be inhibited.

The content aforementioned is illustrated for fully realizing the present invention. However, the present invention may be implemented in many different forms and should not be construed as limited to the embodiments set forth herein; one skilled in the art may modify and vary the embodiments without departing from the spirit and scope of the present invention, therefore, the embodiments should not be construed as the limitation of the claims.

What is claimed is:

1. A method for preventing cancer, comprising the step of: administering a phthalide compound to a subject, wherein the phthalide compound has an effect of increasing oxygen release efficiency of hemoglobin (Hb) in the subject to prevent the oxygenation level of organs, tissues and cells from falling to a level lower than a critical threshold, that is 60% of the physiological oxygen requirements under which the normal cells may turn cancerous.

2. The method of claim 1, wherein the phthalide compound is selected from the group consisting of Z-butylidenephthalide, Z-ligustilide, senkyunolide A, senkyunolide H, senkyunolide I, senkyunolide F, E-butylidenephthalide, E-ligustilide, 3-butylphthalide, 3-butylidene-4-hydrophthalide, 6,7-dihydroxyligustilide and 6,7-epoxyligustilide.

3. The method of claim 1, wherein the phthalide compound has a synergistic effect with 2,3-BPG on hemoglobin (Hb) to increase its oxygen release efficiency.

4. The method of claim 1, wherein the methods for administering the phthalide compound comprises oral administration, injection and inhalation as aerosolized medication.

5. The method of claim 1, which further comprises a step of administering 2,3-BPG to the subject.

6. The method of claim 1, wherein the subject includes a person who has a family history of cancer, who is constantly exposed to carcinogenic environment, and who frequently consumes processed food.

7. The method of claim 1, wherein the oxygenation level of organs, tissues, cells is prevented from reducing to a level lower than 60% of the normal physiological oxygen requirements.
